(19) United States
(12) Patent Application Publication
(10) Pub. No.: US 2026/0028372 A1
(43) Pub. Date: Jan. 29, 2026

(54) PROTEIN EXTRACTION, PROTEIN IDENTIFICATION, AND SPATIAL PROTEOMICS USING PHOTOCLEAVABLE ANCHOR IN SWELLABLE MATERIAL

(71) Applicant: Massachusetts Institute of Technology, Cambridge, MA (US)

(72) Inventors: Camille Marie Mitchell, Cambridge, MA (US); Edward Stuart Boyden, Chestnut Hill, MA (US)

(73) Assignee: Massachusetts Institute of Technology, Cambridge, MA (US)

(21) Appl. No.: 18/998,505

(22) PCT Filed: Jul. 25, 2023
(86) PCT No.: PCT/US2023/070951

§ 371 Date: Jan. 26, 2025

Related U.S. Application Data
(60) Provisional application No. 63/392,294, filed on Jul. 26, 2022.

(51) Int. Cl. C07K 1/14; C07D 207/46

(52) U.S. Cl. CPC C07K 1/145; C07D 207/46

(57) ABSTRACT

The present disclosure provides photocleavable linkers capable of embedding proteins, of biological samples, within swellable materials, methods for extracting at least one protein from a biological sample, and methods for protein identification in biological samples.

### RELATED APPLICATIONS

This application claims the benefit under 35 U.S.C. § 119 (e) of U.S. Provisional application Ser. No. 63/392,294 filed Jul. 26, 2022, the disclosure of which is incorporated by reference herein in its entirety.

### BACKGROUND

In the field of proteomics, there is great interest in developing single-molecule protein detection technologies to improve gold-standard methods, such as mass spectrometry and Edman degradation, that lack in sensitivity and dynamic range (Restrepo-Pérez, L., et. al. (2018). Nature Nanotechnology, 13 (9), 786-796). Protein fingerprinting with fluorescence, or characterization with nanopores, have been developed as ways of sequencing proteins at the single-molecule level (Swaminathan, J., et al., (2018). Nature biotechnology, 36 (11), 1076-1082; Brinkerhoff, H., et al., (2021) Science, 374 (6574), 1509-1513).

Several companies are focusing on making this technology accessible. However, the methods developed are unable to capture the spatial dimension of the biological specimen. For these reasons, immunohistochemistry remains the method of choice for determining the spatial distribution of proteins but is unfortunately limited in its multiplexing potential. These limitations prevent a comprehensive understanding of the role of proteins in cellular processes and how they contribute to dysfunction in disease.

### SUMMARY

The present disclosure relates to protein identification at high spatial resolution in biological samples. Aspects of the present disclosure combine anchoring biomolecules, such as proteins in a biological sample to photocleavable linkers, thereafter, embedding the biological sample (via the photocleavable linkers) within a swellable material (e.g., a polyelectrolyte gel), thereafter expanding the swellable material to physically expand the biological sample, thereafter selectively photocleaving one or more of the linkers, and extracting the untethered protein(s) from the expanded swellable material. In some embodiments, single-molecule protein sequencing may be performed on the extracted protein(s).

By selectively photocleaving proteins from the expanded biological sample, and sequencing the extracted proteins, the spatial location of the proteins of the biological sample may be determined. This spatial location can then be used to understand the role of proteins in cellular processes and how the proteins contribute to dysfunction in disease.

The teachings of the present disclosure have several advantages over existing methods, devices, and materials. Unlike laser microdissection, which has up to 50 μm spatial resolution, the teachings of the present disclosure are able to obtain nanometer range resolution, allowing for the observation of subcellular structures. Moreover, unlike immunohistochemistry, the teachings of the present disclosure do not need a predefined set of protein targets. Additionally, unlike present single molecule protein sequencing methods, the teachings of the present disclosure are able to incorporate the spatial component of a biological sample into single molecule protein sequencing.

A first aspect of the present disclosure relates to a photocleavable linker capable of embedding proteins, a non-limiting example of which are proteins in a biological sample, within a swellable material, the photocleavable linker having the formula:

$$A - B - C$$

where A is a protein-binding moiety, B is a photocleavable moiety, and C is a swellable material-binding moiety. In some embodiments of the first aspect, the protein-binding moiety is capable of binding to an amine group of a protein. In some embodiments of the first aspect, the protein-binding moiety comprises one or more N-hydroxysuccinimide esters (NHS-esters), one or more N,N′-Bis(acryloyl) cystamines, one or more diazirines, one or more epoxides, or a combination thereof. In some embodiments of the first aspect, the protein-binding moiety is selected from the group consisting of N-hydroxysuccinimide ester (NHS-ester), N,N′-Bis(acryloyl) cystamine, a diazirine, and an epoxide. In some embodiments of the first aspect, the photocleavable moiety comprises one or more nitrobenzyls, one or more nitrobenzyl derivatives, one or more azobenzenes, one or more ruthenium (II) caged aminosilanes, one or more coumarins, or a combination thereof. In some embodiments of the first aspect, the one or more nitrobenzyl derivatives comprise one or more ortho-nitrobenzyl esters. In some embodiments of the first aspect, the one or more ruthenium (II) caged aminosilanes comprise one or more ruthenium-bipyridine-triphenylphosphine caged gamma-aminobutyric acids (RuBi-GABAs). In some embodiments of the first aspect, the photocleavable moiety is selected from the group consisting of nitrobenzyl, a nitrobenzyl derivative, azobenzene, ruthenium (II) caged aminosilane, and coumarin. In some embodiments of the first aspect, the swellable material-binding moiety comprises one or more acryloyls, one or more acrylamides, one or more azides, or a combination thereof. In some embodiments of the first aspect, the swellable material-binding moiety is selected from the group consisting of an acryloyl, an acrylamide, and an azide.

A second aspect of the present disclosure relates to a method for extracting at least one protein from a biological sample, where the method comprises: obtaining a biological sample comprising proteins; contacting the biological sample with a photocleavable linker of the first aspect of the present disclosure, resulting in the biological sample having anchored proteins; forming a swellable material around the biological sample having the anchored proteins, resulting in the photocleavable linker binding to the swellable material; expanding the swellable material, thereby separating the anchored proteins; after expanding the swellable material, photocleaving the photocleavable linker, of at least one of the anchored proteins, resulting in at least one untethered protein; and extracting the at least one untethered protein. In some embodiments of the second aspect, the biological sample is a tissue sample. In some embodiments of the second aspect, the tissue sample is less than about 200 μm thick. In some embodiments of the second aspect, the tissue sample is between about 200 μm and about 500 μm thick. In some embodiments of the second aspect, the tissue sample is a brain tissue sample. In some embodiments of the second aspect, the biological sample is a cultured cell and/or cultured tissue. In some embodiments of the second aspect, the swellable material is a swellable polymer. In some embodiments of the second aspect, the swellable polymer is a polyelectrolyte gel. In some embodiments of the second aspect, the expanding comprises isotropically expanding the swellable material. In some embodiments of the second aspect, the photocleaving comprises contacting the photocleavable linker, of the at least one anchored protein, with ultraviolet (UV) light. In some embodiments of the second aspect, the UV light has a wavelength of about 300 nm to about 365 nm. In some embodiments of the second aspect, the UV light has a wavelength of about 365 nm. In some embodiments of the second aspect, the UV light has a wavelength of about 375 nm to about 450 nm. In some embodiments of the second aspect, the UV light has a wavelength of about 427 nm. In some embodiments of the second aspect, the UV light has a wavelength of about 410 nm to about 500 nm. In some embodiments of the second aspect, the UV light has a wavelength of about 460 nm. In some embodiments of the second aspect, the photocleaving comprises contacting the photocleavable linker, of the at least one anchored protein, with visible light. In some embodiments of the second aspect, the photocleaving comprises contacting the photocleavable linker, of the at least one anchored protein, with infrared light. In some embodiments of the second aspect, the method comprises performing the photocleaving using a two-photon microscope. In some embodiments of the second aspect, the two-photon microscope has a lateral resolution of about 0.64 μm. In some embodiments of the second aspect, the two-photon microscope has an axial resolution of about 3.35 μm. In some embodiments of the second aspect, the method comprises performing the photocleaving using a confocal microscope. In some embodiments of the second aspect, the extracting is performed using an electric field. In some embodiments of the second aspect, the extracting is performed using gel electrophoresis. In some embodiments of the second aspect, the extracting is performed using stochastic electrotransport. In some embodiments of the second aspect, the method comprises identifying the at least one extracted protein. In some embodiments of the second aspect, the at least one protein is an untargeted protein. In some embodiments of the second aspect, the at least one extracted protein is an untargeted protein.

A third aspect of the present disclosure relates to a method for protein identification in a biological sample, the method comprising: a method of the second aspect of the present disclosure; and identifying the at least one extracted protein. In some embodiments of the third aspect, identifying the at least one extract protein comprises use of one or more of nanopore sequencing, protein fingerprinting, dynamic protein sequencing, sequential amino acid isolation, and converting information into nucleic acids. In some embodiments of the third aspect, the protein identification is untargeted protein identification. In some embodiments of the third aspect, the protein identification is targeted protein identification.

### DETAILED DESCRIPTION

The present disclosure relates to protein identification at high spatial resolution in biological samples for the purpose of, for example, determining the spatial relationship of proteins within the biological sample. Some embodiments of the present disclosure relate to untargeted protein identification. As used herein, “untargeted protein identification” refers to identifying one or more proteins in a biological sample without processing the biological sample in a manner intended to identify any specific protein(s) therein. Some embodiments of the present disclosure relate to targeted protein identification. As used herein, “targeted protein identification” refers to processing a biological sample to identify one or more specific proteins therein. In some embodiments, proteins of a biological sample may be detectably labeled to enable visualization of all proteins of the biological sample. In some embodiments, particular proteins of a biological sample may be detectably labeled to enable visualization of the particular proteins. One or more proteins of a biological sample may be detectably labeled prior to the biological sample being embedded within a swellable material.

Aspects of the present disclosure combine anchoring proteins of a biological sample to photocleavable linkers, thereafter, embedding the biological sample (via the photocleavable linkers) within a swellable material (e.g., a polyelectrolyte gel), thereafter expanding the swellable material to physically expand the biological sample, thereafter selectively photocleaving one or more of the linkers, and extracting the untethered protein(s) from the expanded swellable material. In some embodiments, single-molecule protein sequencing may be performed on the extracted protein(s).

### Photocleavable Linkers

Aspects of the present disclosure relate to photocleavable linkers capable of embedding proteins of a biological sample, within a swellable material. A photocleavable linker of the present disclosure may have the structure of Formula 1:

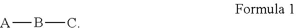

where A is a protein-binding moiety, B is a photocleavable moiety, and C is a swellable material-binding moiety.

As used herein, the terms “bind,” “binding,” and “bound” refer to both covalent interactions and noncovalent interactions. In some embodiments, covalent attachment may be used, but generally all that is required is that the photocleavable linker remain attached to the biological sample. Attachment can occur via hybridization to the biological sample. The term “attach” may be used interchangeably herein with the terms, “anchor(ed),” affix(ed), link(ed), embed(ded), and immobilize(d).

The role of the protein-binding moiety is to bind to protein in a biological sample. The protein-binding moiety may not be selective for any particular protein or class of protein.

The protein-binding moiety may be capable of binding to an amine group of a protein, without being selective for any particular amine group. In other words, the protein-binding moiety may be capable of binding to side chain amine groups of a protein, as well as the N-terminus amine. The protein-binding moiety may be capable of binding to a primary, secondary, and/or tertiary amine.

In some embodiments, the protein-binding moiety may include one or more N-hydroxysuccinimide esters (NHS-esters). In such embodiments, the protein-binding moiety may be capable of binding to a primary amine. Formula 2 below provides a conceptual illustration of a photocleavable linker of the present disclosure where the protein-binding moiety is NHS-ester.

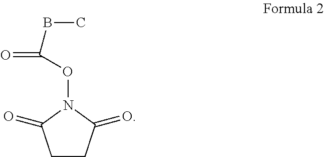

In some embodiments, the protein-binding moiety may include one or more N,N′-Bis(acryloyl) cystamines. In such embodiments, the protein-binding moiety may be capable of binding to cysteine amino acid side chains.

In some embodiments, the protein-binding moiety may include one or more diazirines. Diazirines are a class of organic molecules consisting of a carbon bound to two nitrogen atoms, which are double-bonded to each other, forming a cyclopropane-like ring. Diazirines are photo-activatable chemical groups. Photo-activation of diazirine creates reactive carbene intermediates, which can form covalent bonds through addition reactions with any amino acid side chain or peptide backbone.

In some embodiments, the protein-binding moiety may include one or more epoxides. Epoxides, also known as oxiranes, are three-membered ring structures in which one of the atoms is an oxygen and the other two are carbons. Epoxides react with nucleophile substrates in biological systems, including nucleic acids and amino acids. Guanine, adenine, and cytosine can react with epoxide, as well as thiol, imidazole, amino, carboxyl, and phenol groups present in proteins (e.g., amino acids such as Cys, His, Lys, Asp, Glu, and Tyr). (Cui, Y., et al., bioRxiv (2022) A Multifunctional Anchor for Multimodal Expansion Microscopy. doi.org/10.1101/2022.06.19.496699.)

In some embodiments, the protein-binding moiety includes two or more of N-hydroxysuccinimide ester (NHS-ester), N,N′-Bis(acryloyl) cystamine, a diazirine, and an epoxide. Put another way, the protein-binding moiety may include one or more N-hydroxysuccinimide esters (NHS-esters), one or more N,N′-Bis(acryloyl) cystamines, one or more diazirines, one or more epoxides, or a combination thereof.

In some embodiments, the protein-binding moiety be selected from the group consisting of N-hydroxysuccinimide ester (NHS-ester), N,N′-Bis(acryloyl) cystamine, a diazirine, and an epoxide.

In some embodiments, the photocleavable moiety may include one or more nitrobenzyls.

In some embodiments, the photocleavable moiety may include one or more nitrobenzyl derivatives. Example nitrobenzyl derivatives include, but are not limited to, nitrobenzyl halide (e.g., bromide or chloride), nitrobenzyl alcohol, nitrobenzyl chloroformate, nitrobenzyl pyridine, and nitrobenzyl ester. In some embodiments, the photocleavable moiety may include one or more ortho-nitrobenzyl esters.

In some embodiments, the photocleavable moiety may include one or more azobenzenes. Azobenzene is composed of two phenyl rings linked by a N═N double bond.

In some embodiments, the photocleavable moiety may include one or more ruthenium (II) caged aminosilanes.

In some embodiments, the photocleavable moiety may include one or more ruthenium-bipyridine-triphenylphosphine caged gamma-aminobutyric acids (RuBi-GABAs).

In some embodiments, the photocleavable moiety may include one or more coumarins. Coumarins are phenolic substances composed of used benzene and α-pyrone rings.

In some embodiments, the photocleavable moiety includes two or more of a nitrobenzyl, a nitrobenzyl derivative, an azobenzene, a ruthenium (II) caged aminosilane, and a coumarin. Put another way, the photocleavable moiety may include one or more nitrobenzyls, one or more nitrobenzyl derivatives, one or more azobenzenes, one or more ruthenium (II) caged aminosilanes, one or more coumarins, or a combination thereof.

In some embodiments, the photocleavable moiety is selected from the group consisting of nitrobenzyl, a nitrobenzyl derivative, azobenzene, ruthenium (II) caged aminosilane, and coumarin.

The swellable material-binding moiety may be a physical, biological, or chemical moiety that attaches or crosslinks the biological sample to the swellable material. This may be accomplished by crosslinking the swellable material-binding moiety with the swellable material, such as during or after polymerization (i.e., in situ formation of the swellable material).

In some embodiments, the swellable material-binding moiety may comprise a polymerizable moiety. The swellable material-binding moiety may include, but is not limited to, vinyl or vinyl monomers such as styrene and its derivatives (a non-limiting example of which is divinyl benzene), acrylamide and its derivatives, butadiene, acrylonitrile, vinyl acetate, or acrylates and acrylic acid derivatives. In a non-limiting example, the polymerizable moiety may be an acrylamide modified moiety that may be covalently fixed within a swellable material.

In some embodiments, the swellable material-binding moiety may include one or more acryloyls. Acryloyls are capable of reacting in free-radical-chain-growth-polymerized polyacrylate hydrogel. Formulae 3 and 4 below provide conceptual illustrations of photocleavable linkers of the present disclosure where the swellable material-binding moiety is an acryloyl group. In Formula 4, n is any integer capable of being synthesized.

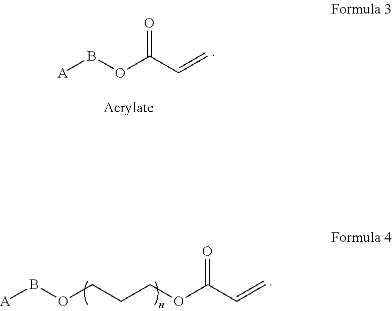

In some embodiments, the swellable material-binding moiety may include one or more acrylamides. Acrylamides are capable of reacting in free-radical-chain-growth-polymerized polyacrylate hydrogel. Formulae 5 and 6 below provide non-limiting examples of conceptual illustrations of photocleavable linkers of the present disclosure where the swellable material-binding moiety is an acrylamide moiety. In Formula 6, n is any integer capable of being synthesized.

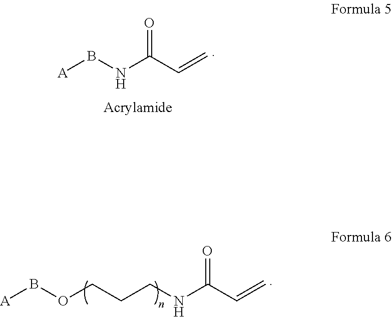

In some embodiments, the swellable material-binding moiety may include one or more azides. Azides are capable of binding to a non-radical polymerized polymer network through click chemistry of tetrahedral monomers. Formula 7 below provides a non-limiting example of a conceptual illustration of a photocleavable linker of the present disclosure where the swellable material-binding moiety is an azide group.

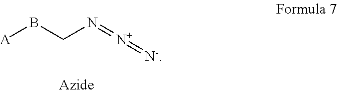

In some embodiments, the swellable material-binding moiety includes two or more of an acryloyl, an acrylamide, and an azide. Put another way, the swellable material-binding moiety may include one or more acryloyls, one or more acrylamides, one or more azides, or a combination thereof. It will be understood that a swellable material-binding moiety that includes two or more of an acryloyl, an acrylamide, and an azide, may in some embodiments comprise (1) one or more acryloyl and one or more acrylamide without including an azide; (2) one or more acryloyl and one or more azide without including an acrylamide; or (3) one or more acrylamide and one or more azide without including an acryloyl. It will be understood that a swellable material-binding moiety that includes two or more of an acryloyl, an acrylamide, and an azide, may in some embodiments comprise (4) two more acryloyls without including an acrylamide or an azide; (5) two or more acrylamides without including an acryloyl or an azide; or (6) two or more azides, without including an acrylamide or an acryloyl.

In some embodiments, the swellable material-binding moiety is selected from the group consisting of an acryloyl, an acrylamide, and an azide.

In some embodiments, the protein-binding moiety may be NHS-ester and the photocleavable moiety may be ortho-nitrobenzyl ester.

In some embodiments, the photocleavable linker may be 1-(1-(4-(acryloyloxy)-5-methoxy-2-nitrophenyl)ethyl) 7-(2,5-dioxopyrrolidin-1-yl) heptanedioate, having the chemical structure:

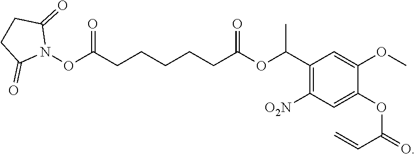 [SMILES: C=CC(=O)Oc1cc([N+](=O)[O-])c(C(C)OC(=O)CCCCCC(=O)ON2C(=O)CCC2=O)cc1OC]

### Swellable Materials

As used herein, “swellable material” generally refers to a material that expands when contacted with a liquid, such as water or other solvent. Additionally, or alternatively, the swellable material can be expanded by any other means known to one of skill in the art. In some embodiments, the swellable material uniformly expands in three dimensions (i.e., isotropically). Additionally, or alternatively, the material is transparent such that, upon expansion, light can pass through the sample. In some embodiments, the swellable material may be a swellable polymer.

The swellable material may be formed in situ from precursors thereof. For example, though not intended to be limiting, in some embodiments, embedding the biological sample in the swellable material includes permeating the biological sample with a composition including one or more precursors of the swellable material, and polymerizing and/or crosslinking the precursors to form the swellable material. In this manner the biological sample is embedded in the swellable material.

As used herein, “precursors of swellable material” means hydrophilic monomers, prepolymers, or polymers that can be crosslinked, or “polymerized,” to form a three-dimensional (3D) network. Precursors can also comprise polymerization initiators and crosslinkers.

In some embodiments, the swellable material is a polyelectrolyte. In some embodiments, the swellable material is polyacrylate or polyacrylamide and copolymers or crosslinked copolymers thereof.

In some embodiments, one or more polymerizable materials, monomers, or oligomers can be used, such as monomers selected from the group consisting of water-soluble groups containing a polymerizable ethylenically unsaturated group. Monomers or oligomers can include one or more substituted or unsubstituted methacrylates, acrylates, acrylamides, methacrylamides, vinylalcohols, vinylamines, allylamines, allylalcohols, including divinylic crosslinkers thereof (e.g., N, N-alkylene bisacrylamides).

In some embodiments, the precursor of the swellable material may include at least one polyelectrolyte monomer and a covalent crosslinker. In some embodiments, the swellable material may be a hydrogel. In some embodiments, the hydrogel is a polyacrylate hydrogel. In some embodiments, the precursor of the swellable material includes acrylate, acrylamide, and a crosslinker selected from N,N-methylenebisacrylamide (BIS), N,N′-(1,2-Dihydroxythylene)bisacryalmide) (DHEBA); and N,N′-Bis(acryloyl)cystamine (BAC).

The precursors of the swellable polymer may be delivered to the biological sample by any convenient method including, but not limited to, permeating, perfusing, infusing, soaking, adding or other intermixing of the biological sample with the precursors of swellable material. In this manner, the biological sample is saturated with precursors of the swellable material, which flow between and around biomolecules (e.g., proteins) throughout the biological sample.

Following permeating the biological sample, the swellable polymer precursors may be polymerized (i.e., covalently or physically crosslinked) to form a polymer network. The polymer network is formed within and throughout the biological sample. In this manner, the biological sample is saturated with the swellable material, which flows between and around biomolecules (a non-limiting example of which are proteins) throughout the biological sample.

Polymerization may be by any method including, but not limited to, thermal crosslinking, chemical crosslinking, physical crosslinking, ionic crosslinking, photo-crosslinking, irradiative crosslinking (e.g., x-ray, electron beam), and the like, and may be selected based on the type of hydrogel used and knowledge in the art. In some embodiments, the polymer is a hydrogel. Once polymerized, a polymer-embedded biological sample is formed.

In some embodiments, the swellable material is polyacrylate and copolymers or crosslinked copolymers thereof. As a non-limiting example, if the biological sample is to be embedded in sodium polyacrylate, a solution comprising the monomers sodium acrylate and acrylamide, and a crosslinker selected from N,N-methylenebisacrylamide (BIS), N,N′-(1,2-Dihydroxythylene)bisacrylamide), and (DHEBA) N,N′-Bis(acryloyl)cystamine (BAC), may be perfused throughout the biological sample.

In some embodiments, the swellable material is a swellable polymer or hydrogel. The hydrogel may be a polyelectrolyte hydrogel. The polyelectrolyte may be a polyacrylate.

By embedding a biological sample in a swellable polymer that physically supports the ultrastructure of the biological sample, the three-dimensional distribution of the biomolecules (non-limiting examples of which are proteins) in the biological sample are preserved, resulting in the biomolecules being secured or anchored in the polymer network.

After the biological sample has been anchored to the swellable material, the biological sample may be subjected to a disruption of the endogenous biological molecules or the physical structure of the biological sample, leaving the macromolecules that preserve the information of the biological molecules intact and anchored to the swellable polymer. In this way, the mechanical properties of the biological sample-swellable material complex are rendered more spatially uniform, allowing greater and more consistent isotropic expansion.

The disruption of the endogenous physical structure of the biological sample, or of the endogenous biological molecules of the biological sample, generally refers to the mechanical, physical, chemical, biochemical, or (protease) enzymatic digestion, disruption, or break up of the biological sample so that it will not resist expansion. In some embodiments, a detergent is used to homogenize the biological sample-swellable material complex.

In some embodiments, the detergent is in a buffer having a pH from about 4 to about 12. Any suitable buffer agent can be used including, but not limited to, Tris, citrate, phosphate, bicarbonate, MOPS, borate, TAPS, bicine, Tricine, HEPES, TES, and MES.

In some embodiments, the buffer includes a detergent, a metal ion chelator, and/or a nonionic surfactant. In some embodiments, the buffer includes about 20% w/v of a detergent in a buffer having a pH between about 4 and about 12, the buffer including about 5 mM to about 100 mM of a metal ion chelator, about 0.1% to about 1.0% of a nonionic surfactant, and antioxidant in the range 10-100 mM. In some embodiments, the biological sample is incubated in the buffer for about 1-2 hrs at 120° C. in an autoclave.

In some embodiments, prior to the detergent treating step, the biological sample is treated with a collagenase enzyme solution in the range 300-1200 U/ml at 37° C. temperature for 1-6 hrs in a Hanks Balance Salt Solution buffer with calcium, and any suitable antigen retrieval process known to one of skill in the art. Subsequently, the biological sample may be treated using a buffer including about 20% w/v of a detergent in a buffer having a pH between about 4 and about 12, the buffer including about 5 mM to about 100 mM of a metal ion chelator, about 0.1% to about 1.0% of a nonionic surfactant, and antioxidant in the range 10-100 mM.

Information herein describes ranges of amounts of detergent, metal ion chelator, nonionic surfactant, and antioxidants from which an amount of each of detergent, metal ion chelator, nonionic surfactant, and/or antioxidant may be selected and included in the buffer. In some embodiments, a buffer used in a method of the invention includes a combination of detergent, metal ion chelator, and/or nonionic surfactant, each in an amount independently selected from a range of detergent, metal ion chelator, nonionic surfactant, and antioxidant set forth below herein and mixed together in the buffer. As used herein, the term independently selected used in reference to components in the buffer, means each of a detergent, a metal chelator, a nonionic surfactant, and an antioxidant can be selected separately from the other components. As non-limiting examples, a buffer may be prepared that comprises SDS as the detergent, EDTA as a chelating agent, Triton X-100 as a nonionic surfactant, and β-mercaptoethanol as an antioxidant and another buffer may be prepared that comprises SDS as the detergent, BAPTA as a chelating agent, Triton X-100 as a nonionic surfactant, and β-mercaptoethanol as an antioxidant.

Detergents are well known to those of skill in the art. An example detergent is sodium dodecyl sulfate (SDS). In some embodiments, the buffer includes about 20% of a detergent. In some embodiments, the buffer includes a detergent at a percentage in the buffer of between about 5-20%. In some embodiments, the buffer includes a detergent at a percentage in the buffer of between 5% and 10%, between 10% and 15%, or between 15% and 20%.

Chelating agents are well known to those of skill in the art. Example chelating agents include, but are not limited to, EDTA, EGTA, EDDHA, EDDS, BAPTA and DOTA. In some embodiments, the buffer includes about 5 mM to about 100 mM of a metal ion chelator. In some embodiments, the buffer includes about 5 mM to about 75 mM of a metal ion chelator. In some embodiments, the buffer includes about 5 mM to about 50 mM of a metal ion chelator. In some embodiments, the buffer comprises a metal ion chelator at a concentration in the buffer that is at least one of between: 15 mM and 100 mM; 15 mM and 90 mM; 15 mM and 80 mM; 15 mM and 70 mM; 15 mM and 60 mM; 15 mM and 50 mM; 15 mM and 40 mM; 15 mM and 30 mM; and 15 mM and 20 mM. In some embodiments, the buffer comprises a metal ion chelator at a concentration in the buffer that is at least one of between: 25 mM and 100 mM; 25 mM and 90 mM; 25 mM and 80 mM; 25 mM and 70 mM; 25 mM and 60 mM; 25 mM and 50 mM; 25 mM and 40 mM; and 25 mM and 30 mM. In some embodiments, the buffer comprises a metal ion chelator at a concentration in the buffer that is at least one of between: 35 mM and 100 mM; 35 mM and 90 mM; 35 mM and 80 mM; 35 mM and 70 mM; 35 mM and 60 mM; 35 mM and 50 mM; and 35 mM and 40 mM. In some embodiments, the buffer comprises a metal ion chelator at a concentration in the buffer that is at least one of between: 45 mM and 100 mM; 45 mM and 90 mM; 45 mM and 80 mM; 45 mM and 70 mM; 45 mM and 60 mM; and 45 mM and 50 mM. In some embodiments, the buffer comprises a metal ion chelator at a concentration in the buffer that is at least one of between: 55 mM and 100 mM; 55 mM and 90 mM; 55 mM and 80 mM; 55 mM and 70 mM; and 55 mM and 60 mM. In some embodiments, the buffer comprises a metal ion chelator at a concentration in the buffer that is at least one of between: 65 mM and 100 mM; 65 mM and 90 mM; 65 mM and 80 mM; and 65 mM and 70 mM. In some embodiments, the buffer comprises a metal ion chelator at a concentration in the buffer that is at least one of between: 75 mM and 100 mM; 75 mM and 90 mM; and 75 mM and 80 mM. In some embodiments, the buffer comprises a metal ion chelator at a concentration in the buffer that is at least one of between: 85 mM and 100 mM; and 85 mM and 90 mM. In some embodiments, the buffer comprises a metal ion chelator at a concentration in the buffer that is between: 95 mM and 100 mM.

Nonionic surfactants are well known to those of skill in the art. Example nonionic surfactants include, but are not limited to, Triton X-100, Tween 20, Tween 80, Sorbitan, Polysorbate 20, Polysorbate 80, PEG, Decyl glucoside, Decyl polyglucose and cocamide DEA. In some embodiments, the buffer includes about 0.1% to about 1.0% nonionic surfactant. In some embodiments, the buffer includes about 0.1% to about 0.75% nonionic surfactant. In some embodiments, the buffer includes about 0.1% to about 0.5% nonionic surfactant. In some embodiments, the buffer includes about 0.1% to about 0.3% nonionic surfactant. In some embodiments, the buffer comprises a nonionic surfactant % that is at least one of between: 2% and 1.0%; 3% and 1.0%; 0.4% and 1.0%; 0.5% and 1.0%; 0.6% and 1.0%; 0.7% and 1.0%; 0.8% and 1.0%; and 0.9% and 1.0%. In some embodiments, the buffer comprises a nonionic surfactant % that is at least one of between: 0.3% and 0.9%; 0.3% and 0.8%; 0.3% and 0.7%; 0.3% and 0.6%; 0.3% and 0.0.5%; and 0.3% and 0.4%. In some embodiments, the buffer comprises a nonionic surfactant % that is at least one of between: 0.4% and 0.9%; 0.4% and 0.8%; 0.4% and 0.7%; 0.4% and 0.6%; and 0.4% and 0.5%. In some embodiments, the buffer comprises a nonionic surfactant % that is at least one of between 0.5% and 0.9%; 0.5% and 0.8%; 0.5% and 0.7%; and 0.5% and 0.6%. In some embodiments, the buffer comprises a nonionic surfactant % that is at least one of between 0.6% and 0.0.9%; 0.6% and 0.8%; and 0.6%, and 0.7%. In some embodiments, the buffer comprises a nonionic surfactant % that is at least one of between 0.7% and 0.9%; and 0.7% and 0.8%. In some embodiments, the buffer comprises a nonionic surfactant % that is between 0.8% and 0.9%.

Antioxidants are well known to those of skill in the art. Example antioxidants include, but are not limited to, β-mercaptoethanol and dithiothretiol. In some embodiments, the buffer includes about 10-100 mM of an antioxidant. In some embodiments, the buffer includes about 10 mM to 90 mM of an antioxidant. In some embodiments, the buffer comprises an antioxidant at a concentration in the buffer that is at least one of between 20 mM and 90 mM; 20 mM and 80 mM; 20 mM and 70 mM; 20 mM and 60 mM; 20 mM and 50 mM; 20 mM and 40 mM; and 20 mM and 30 mM. In some embodiments, the buffer comprises an antioxidant at a concentration in the buffer that is at least one of between: 30 mM and 90 mM; 30 mM and 80 mM; 30 mM and 70 mM; 30 mM and 60 mM; 30 mM and 50 mM; and 30 mM and 40 mM. In some embodiments, the buffer comprises an antioxidant at a concentration in the buffer that is at least one of between: 40 mM and 90 mM; 40 mM and 80 mM; 40 mM and 70 mM; 40 mM and 60 mM; and 40 mM and 50 mM. In some embodiments, the buffer comprises an antioxidant at a concentration in the buffer that is at least one of between: 50 mM and 90 mM; 50 mM and 80 mM; 50 mM and 70 mM; and 50 mM and 60 mM. In some embodiments, the buffer comprises an antioxidant at a concentration in the buffer that is at least one of between: 60 mM and 90 mM; 60 mM and 80 mM; and 60 mM and 70 mM. In some embodiments, the buffer comprises an antioxidant at a concentration in the buffer that is at least one of between: 70 mM and 90 mM; and 70 mM and 80 mM. In some embodiments, the buffer comprises an antioxidant at a concentration in the buffer that is between: 80 mM and 90 mM. In each instance, the recited range is inclusive of the first and last numbers in the range.

It is preferable that the disruption does not impact the structure of the swellable material but disrupts the structure of the biological sample. Thus, the disruption should be substantially inert to the swellable material. The degree of digestion can be sufficient to compromise the integrity of the mechanical structure of the biological sample, or it can be complete to the extent that the biological sample-swellable material complex is rendered substantially free of the biological sample.

In some embodiments, the physical disruption of the biological sample is accomplished by a mild disruption treatment that minimizes damage to the individual proteins, allowing staining and other treatments on the proteins to be carried out after expansion. In some embodiments, such milder treatment is performed by using LyC or trypsin. In some embodiments, such milder treatment is performed by heating the biological sample. In some embodiments, heating the biological sample is performed by autoclaving the sample.

The biological sample-swellable material complex is then expanded by, for example, contacting the swellable material with a solvent or liquid that is then absorbed by the swellable material and causes swelling. Where the swellable material is water swellable, an aqueous solution can be used. The swelling of the swellable material results in the biological sample itself expanding (e.g., becoming larger). This is because the swellable material is embedded throughout the biological sample and, therefore, as the swellable material grows, it expands and causes the anchored proteins to pull apart (i.e., move away) from each. In some embodiments, the swellable material expands isotropically; therefore, the anchored proteins retain the relative spatial location within the biological sample.

In some embodiments, the biological sample may be iteratively enlarged. For example, after the biological sample is enlarged as described above, the biological sample may undergo one or more additional iterations of the above expansions.

In some embodiments, the biological sample and each iterative enlargement thereof may be permeated with one or more monomers or precursors or a solution comprising one or more monomers or precursors that are then reacted (e.g., polymerized) to form a swellable or non-swellable material depending on what step of the method is being performed.

“Re-embedding” the expanded sample in a non-swellable material may include permeating (such as perfusing, infusing, soaking, adding, or other intermixing) the expanded sample with the non-swellable material, for example by adding precursors thereof. Alternatively, or additionally, embedding the enlarged sample in a non-swellable material may include permeating one or more monomers or other precursors throughout the expanded sample and polymerizing and/or crosslinking the monomers or precursors to form the non-swellable material or polymer. Embedding the expanded sample in a non-swellable material may prevents conformational changes (e.g., shrinkage) during following steps despite salt concentration variation. The non-swellable material can be a charge-neutral hydrogel. For example, it can be polyacrylamide hydrogel, composed of acrylamide monomers, bisacrylamide crosslinker, ammonium persulfate (APS) initiator and tetramethylethylenediamine (TEMED) accelerator.

In some embodiments, different swellable materials may be used to expand the same biological sample.

In some embodiments, a single expansion of a biological sample may achieve an about 4× linear expansion factor of the biological sample. In some embodiments, iterative expansion of a biological sample may achieve an about 20× linear expansion factor.

In some embodiments, the swellable material may be assembled using non-radical terminal linking chemistry by tetrahedral monomer linking, resulting in a highly homogeneous network structure. Other chemistry protocols could also be employed to assemble the swellable material.

### Detectable Labels

In some embodiments, the biological sample is labeled with a detectable label. Typically, the label will bind chemically (e.g., covalently, hydrogen bonding or ionic bonding) to a biomolecule of the sample, or a component thereof. The detectable label can be selective for a specific target (e.g., for proteins), as can be accomplished with an antibody or other target-specific binder. The detectable label may comprise a visible component, as is typical of a dye or fluorescent molecule; however, any signaling means used by the label is also contemplated. A fluorescently labeled biological sample, for example, is a biological sample labeled through techniques such as, but not limited to, immunofluorescence, immunohistochemical, or immunocytochemical staining. In some embodiments, the detectable label is chemically attached to the biological sample, or a targeted component thereof (such as proteins in the biological sample). The labeled sample may furthermore include more than one label. For example, each label can have a particular or distinguishable fluorescent property (e.g., distinguishable excitation and emission wavelengths).

### Photocleaving of Photocleavable Linkers

Aspects of the present disclosure relate to photocleaving photocleavable linkers in an expanded swellable material. The wavelength used to photocleave a linker is dependent on the makeup of the photocleavable moiety of the linker.

In some embodiments, a photocleavable linker may be photocleaved using ultraviolet (UV) light.

In some embodiments, the UV light may have a wavelength of about 300 nm to about 365 nm. In some embodiments, the UV light may have a wavelength of about 365 nm. For example, UV light of about 300 nm to about 365 nm, or UV light of about 365 nm, may be used when the photocleavable moiety includes nitrobenzyl, and more specifically ortho-nitrobenzyl ester.

As non-limiting examples, in some embodiments, when the photocleavable moiety includes nitrobenzyl, or in some embodiments, ortho-nitrobenzyl ester, a UV light between about 300 nm and about 365 nm is used; in some embodiments, a UV light between 310 nm and 365 nm is used; in some embodiments, a UV light between 310 nm and 355 nm is used; in some embodiments, a UV light between 310 nm and 345 nm is used; in some embodiments a UV light between 310 nm and 335 nm is used; in some embodiments a UV light between 310 nm and 325 nm is used, with each stated range including the end values of the stated range. As a nonlimiting example, in some embodiments, a UV light of about 365 nm, is used when the photocleavable moiety includes nitrobenzyl, or in some embodiments, ortho-nitrobenzyl ester. In some embodiments, a UV light of about 300 nm, 305 nm, 310 nm, 315 nm, 320 nm, 325 nm, 330 nm, 335 nm, 340 nm, 345 nm, 350 nm, 355 nm, 360 nm, or 365 nm may used in an embodiment of a method of the invention.

In some embodiments, the UV light may have a wavelength of about 375 nm to about 450 nm. In some embodiments, the UV light may have a wavelength of about 427 nm. As non-limiting examples, in some embodiments, when the photocleavable moiety includes azobenzene, a UV light between about 375 nm and about 450 nm is used; in some embodiments, a UV light between 385 nm and 450 nm is used; in some embodiments, a UV light between 400 nm and 450 nm is used; in some embodiments, a UV light between 400 nm and 430 nm is used; in some embodiments a UV light between 425 nm and 445 nm is used; in some embodiments a UV light between 425 nm and 430 nm is used, with each stated range including the end values of the stated range. As a nonlimiting example, in some embodiments, a UV light of about 427 nm, is used when the photocleavable moiety includes azobenzene. In some embodiments, a UV light of about 375 nm, 380 nm, 385 nm, 390 nm, 395 nm, 400 nm, 405 nm, 410 nm, 415 nm, 420 nm, 425 nm, 430 nm, 435 nm, 440 nm, 445 nm, or 450 nm may be used in an embodiment of a method of the invention.

In some embodiments, the UV light may have a wavelength of about 410 nm to about 500 nm. In some embodiments, the UV light may have a wavelength of about 460 nm. As non-limiting examples, in some embodiments, when the photocleavable moiety includes Ruthenium (II), a UV light between about 420 nm and 490 nm is used; in some embodiments, a UV light between 430 nm and 480 nm is used; in some embodiments, a UV light between 440 nm and 470 nm is used; in some embodiments, a UV light between 450 nm and 465 nm is used; in some embodiments a UV light between 455 nm and 465 nm is used; in some embodiments with each stated range including the end values of the stated range. As a non-limiting example, UV light of about 410 nm to about 500 nm, or UV light of about 460 nm, may be used when the photocleavable moiety includes Ruthenium (II). In some embodiments, a UV light of about 410 nm, 415 nm, 420 nm, 425 nm, 430 nm, 435 nm, 440 nm, 445 nm, 450 nm, 455 nm, 460 nm, 465 nm, 470 nm, 475 nm, 480 nm, 485 nm, 490 nm, 495 nm, or 500 nm may be used in an embodiment of a method of the invention.

In some embodiments, a photocleavable linker may be photocleaved using visible light, for example when the photocleavable moiety include RuBi-GABA.

In some embodiments, a photocleavable linker may be photocleaved using infrared light.

In some embodiments, a photocleavable linker may be photocleaved using a two-photon microscope. The lateral resolution of the two-photon microscope may depend on how expanded the biological sample is. As a non-limiting example, in some embodiments, the two-photon microscope may have a lateral resolution of about 0.64 μm. Likewise, the axial resolution of the two-photon microscope may depend on how expanded the biological sample is. As a non-limiting example, in some embodiments, the two-photon microscope may have an axial resolution of about 3.35 μm. If the swellable material leads to an about 20× linear expansion of the biological sample, a sample laser pulse of the two-photon microscope may cleave any proteins about 32 nm or less away from each other prior to expansion. Two-photon microscopy may be a used for photocleaving in at least some embodiments, because it prevents scattered excitation light from cleaving proteins out of the plane of focus.

In some embodiments, a photocleavable linker may be photocleaved using a confocal microscope. However, it is noted that a confocal microscope cannot restrict excitation to a relatively tiny focal volume in thick biological samples (e.g., about 1 mm), as can a two-photon microscopy. Out of focus excitation of a confocal microscope may reduce the spatial resolution of protein cleaving.

Protein Extraction from Expanded Swellable Material

Aspects of the present disclosure relate to extracting proteins from expanded swellable material. As disclosed elsewhere herein, following expansion of swellable material, one or more proteins may be untethered from the expanded swellable material by photocleaving the linker(s) anchoring the protein(s) to the expanded swellable material. Once untethered, the protein(s) is free to migrate along an electric field for extraction, whereas still-tethered proteins that are still anchored to the gel are locked into place.

In some embodiments, protein may be extracted from expanded swellable material using gel electrophoresis. This technique separates the negatively charged proteins by applying an electric field.

Alternatively, proteins could be separated from the expanded swellable material using other methods. One such method includes stochastic electrotransport, which can enhance the diffusivity of electromobile proteins that are unbound to the expanded swellable material and prevent displacement of the anchored proteins. This method relies on a rotational electric field that allows dispersion of highly electromobile proteins.

Example Method for Extracting at Least One Protein from a Biological Sample

Aspects of the present disclosure relates to methods for extracting at least one protein from a biological sample. An example method includes obtaining a biological sample including proteins; contacting the biological sample with a photocleavable linker, resulting in the biological sample having anchored proteins; forming a swellable material around the biological sample having the anchored proteins, resulting in the photocleavable linker binding to the swellable material; expanding the swellable material, thereby separating the anchored proteins; after expanding the swellable material, photocleaving the photocleavable linker, of at least one of the anchored proteins, resulting in at least one untethered protein; and extracting the at least one untethered protein.

By iteratively photocleaving and extracting proteins from the expanded biological sample, the spatial location of the proteins in the biological sample can be determined.

### Protein Identification

Aspects of the present disclosure relate to performing protein identification of proteins extracted from a biological sample. For example, at least one protein may be extracted from a biological sample using the method described above, and the at least one extracted protein may be identified.

Any protein identification technique known or not yet known may be used. The present disclosure is not intended to be bound to any particular protein identification technique.

In some embodiments, nanopore sequencing may be used. Protein sequencing with nanopores relies on directing proteins into a membrane-embedded pore, and reading-out ion currents across the membrane as the different amino acids pass through. Nanopore sequencing is able to detect single-amino acid substitutions within individual peptides. Proteins could be directed into the nanopore after photocleavage from the swellable material using an electric field as described herein above.

In some embodiments, protein fingerprinting may be used. Peptide fingerprinting with fluorosequencing can be used to determine the identity of a parent protein with sparse information, followed by its mapping onto a database. The read-out is based on Edman sequencing of fluorescently labeled lysine and cysteine amino acids that are sequentially cleaved off and imaged under a TIRF microscope. Other protein fingerprinting methods include using Förster Resonance Energy Transfer (FRET) that identifies proteins based on the FRET efficiencies between protein-bound DNA strands, or a donor fluorophore-labeled protease that scans peptides with acceptor-labeled amino acids.

In some embodiments, dynamic protein sequencing may be used. Dynamic sequencing involves binding a set of N-terminal amino acid binders (NAABs) to the N-terminal amino acid and peptides are simultaneously cleaved using aminopeptidases.

Fluorescence intensity, lifetime, and binding kinetics of the multiple different binders onto the N-terminal amino acid of the peptides are then recorded using an integrated semiconductor chip. In some instances, the N-terminal amino acid may be isolated to prevent interference of subsequent residues on the binding properties of NAABs. After isolation of the N-terminal amino acid, binders can be made to be specific to each amino acid rather than having to use a set of NAABs to determine its identity.

In some embodiments, sequential amino acid isolation may be used. In some embodiments, converting information into nucleic acids may be used.

### Biological Samples and Biomolecules

In some embodiments, a biological sample is examined to identify one or more biomolecules present in the sample. The term “biological sample” is used herein in a broad sense and is intended to include sources that contain one or more biomolecules. As used herein, the term “biomolecule” is a molecule comprising one or more of a protein, a carbohydrate, a lipid, a nucleic acid, or any components or combinations thereof. In some embodiments, a biomolecule comprises a protein. In some embodiments, a biomolecule is a protein.

In some embodiments, a biological sample comprises a tissue sample, a non-limiting example of which is an organ tissue sample. A biological sample can be obtained, for example, from autopsy, biopsy, or from surgery. It can be a solid tissue such as, but not limited to, parenchyme, connective or fatty tissue, heart or skeletal muscle, smooth muscle, skin, brain, nerve, kidney, liver, spleen, breast, carcinoma (e.g. bowel, nasopharynx, breast, lung, stomach etc.), cartilage, lymphoma, meningioma, pancreas, embryonic, placenta, prostate, intestine, colon, skin, testis, thymus, tonsil, umbilical cord, or uterus. In some embodiments, a tissue is obtained or derived from a benign tumor or from a malignant tumor. In some embodiments, a tissue sample comprises a cancerous tissue and/or a precancerous tissue.

In some embodiments, a biological sample comprises one or more of a biological fluid, non-limiting examples of which are blood, serum, lymph, urine, saliva, mucus, etc.

A biological sample may be collected (obtained) from a living subject (e.g., a biopsy sample) or may be collected (obtained) from a dead subject (e.g., an autopsy or necrospsy sample). A biological sample may be obtained from a cell culture and/or obtained from an organ culture. In some embodiments, the biological sample comprises a cultured cell.

It will be understood that, optionally, a biological sample may be processed prior to use in certain embodiments of a method of the invention. For example, though not intended to be limiting, a tissue sample may be sectioned, a blood sample may be centrifuged and separated into fractions of which one or more may be used as a biological sample in a method of the invention. Other art-known processing may be used to prepare a biological sample for use in certain embodiments of methods of the invention. In some embodiments a biological sample is a fixed biological sample. Materials obtained from clinical or forensic settings are also within the intended meaning of the term biological sample.

A biological sample may comprise any cell or tissue type, for example but not limited to hematopoietic, neural (central or peripheral), glial, mesenchymal, cutaneous, mucosal, stromal, muscle (skeletal, cardiac, or smooth), spleen, reticulo-endothelial, epithelial, endothelial, hepatic, kidney, pancreatic, gastrointestinal, pulmonary, fibroblast, and other types. Illustrative biological samples include, but are not limited to, tissues of the central nervous system. Other illustrative biological samples include cells and liquid samples. In some embodiments, a biological sample comprises a cultured tissue and/or a cultured cell.

In some embodiments, a biological sample is obtained from an organ (e.g., the whole brain of a rodent) or a portion of an organ or tissue, (e.g., a biopsy of a transplanted tissue). For example, an organ may be obtained and sectioned, with a section of the organ considered a biological sample. In some embodiments, a biological sample is considered as a solid, for example an organ or portion thereof, or a tissue mass. In certain embodiments of methods of the invention a “solid” tissue or organ is sectioned to reduce its thickness prior to being assessed with a method of the invention. In some embodiments, a biological sample comprises a tissue or organ sample having a thickness suitable for use in a method of the invention. In some embodiments a biological sample comprises a tissue or organ sample that is sectioned or otherwise altered to reduce its thickness. In some embodiments, a biological sample may be a tissue sample that is less than about 200 μm thick. In some embodiments, a biological sample comprises a tissue sample that is between 50 μm and 200 μm thick. In some embodiments, a biological sample comprises a tissue sample that is between 100 μm and 200 μm thick. In some embodiments, a biological sample comprises a tissue sample that is between 150 μm and 200 μm thick. In some embodiments, the biological sample comprises a tissue sample that is between about 200 μm and about 500 μm thick. In some embodiments, the biological sample comprises a tissue sample that is between about 250 μm and about 500 μm thick. In some embodiments, the biological sample comprises a tissue sample that is between about 300 μm and about 500 μm thick. In some embodiments, the biological sample comprises a tissue sample that is between about 400 μm and about 500 μm thick. In some embodiments, the biological sample comprises a tissue sample that is between about 200 μm and about 400 μm thick. In some embodiments, the biological sample comprises a tissue sample that is between about 200 μm and about 300 μm thick. In some embodiments, the biological sample comprises a tissue sample that is between about 300 μm and about 500 μm thick. In some embodiments, the biological sample comprises a tissue sample that is between about 3200 μm and about 500 μm thick. In some embodiments, the biological sample comprises a tissue sample that is between about 400 μm and about 500 μm thick.

A biological sample may be collected and processed using the methods and systems described herein and subjected to analysis immediately following processing, or may be preserved (for example, but not limited to snap frozen, chemically fixed, etc.) and subjected to analysis at a future time (e.g., after short-term or extended storage). In some embodiments, a biological sample may be a previously preserved sample such as, for example, a preserved sample from a tissue bank, a non-limiting example of which is a preserved sample of a human brain obtained from a tissue collection program. In some embodiments, a biological sample may be processed and cleared to remove a plurality of cellular components (e.g., lipids), and then stored and for future analysis using a method of the invention.

### Biological Sample Sources

In some embodiments, a biological sample is obtained or derived from a human, animal, or plant. In some embodiments, the biological sample is derived from a human. The term “derived” as used herein in relation to a biological sample, means a sample that originated from a subject and after it is obtained from the subject, has been cultured, transformed, transplanted, grafted, or otherwise altered prior to being assessed using an embodiment of a method of the invention. In some embodiments, a biological sample may comprise one or more engineered cells and/or an engineered tissue. In some embodiments, a biological sample comprises one or more cells obtained from a cell culture. As a non-limiting example, biological sample may be obtained from a cultured adherent cell line such as, but not limited to: HEK and/or COS7.

A biological sample of the present disclosure may be obtained or derived from any plant or animal, including, but not limited to, a wild-type plant or animal, a mutant plant or animal, and a genetically modified plant or animal, a plant or animal obtained from the wild, and a plant or animal grown and/or produced and/or maintained in captivity. Non-limiting examples of animals from which a biological sample may be obtained or derived are vertebrates, including but not limited to mammalian vertebrates, such as but not limited to equine, bovine, ovine, canine, feline, and murine animals. In some embodiments, a biological sample used in an embodiment of a method of the invention is a rodent, a non-human primate, or a human. In some instances, a biological sample may be obtained or derived from a transgenic subject such as, though not limited to a transgenic mouse. Non-limiting examples of an animal from which a biological sample may be obtained or derived are non-mammalian vertebrates, including but not limited to birds, reptiles, amphibians, and fish. Additional non-limiting examples of an animal from which a biological sample may be obtained or derived are invertebrates, such as, but not limited to an insect, a worm, Drosophila, a nematode, etc. In some embodiments, a biological sample comprises a prokaryotic organism. In certain embodiments, a biological sample comprises one or cells of a eukaryotic organism.

In some instances, a biological sample is obtained or derived from a subject known to suffer from a disease or condition. In a non-limiting example, a biological sample may comprise brain tissue from a subject known to have autism. In other instances, a biological sample may be brain tissue obtained from a subject who does not have autism. In another non-limiting example, a biological sample may comprise cultured tissue, originally obtained from a transgenic rodent. In other instances, a biological sample may be a tissue obtained from a subject with a cancer. Methods of the invention can be used to identify biomolecules present in a biological sample and the results compared with results from a control. It will be understood that embodiments of methods of the invention can be used to identify and assess biomolecules present in a sample, optionally compared with a control sample, to assess one or more of status of a disease or condition, efficacy of a treatment for a disease or condition, a genetic change, a developmental status, etc.

### Controls

It will be understood that results obtained from use of a method of the invention to assess one or more biomolecules in a biological sample obtained from a subject, can be compared with a control. In some embodiments a control is a “normal” control, meaning the control biological sample does not have a disease or condition present in the subject. In some embodiments, a control may be a result obtained from a subject prior, or subsequent, to treatment of the subject for a disease or condition.

In some embodiments, a method of the invention is used to assess a biological sample obtained from a subject (also referred to herein as a “test sample”) known to have a disease or condition and the results from the test sample are compared with results from a control biological sample assessed using the method of the invention. Comparison of results of test and control samples are used to identify one or more changes in a cell, tissue, and/or biomolecule associated with (e.g., result from) the disease or condition in the subject. Similarly, methods of the invention can be used to assess efficacy of a therapeutic agent or strategy administered to a cell, tissue, and/or subject to treat a disease or condition. In some embodiments, a control may be results obtained using a method of the invention to assess a first biological sample obtained from a subject, tissue, and/or cell having a disease or condition. Results of the first biological sample may be compared to results obtained for a second biological sample obtained from the subject, tissue, and/or cell following administration of a therapeutic agent or strategy to treat the disease or condition. Results of the first biological sample may serve as a control for the second biological sample and the comparison used to determine a level of efficacy of the therapeutic agent or strategy. Those in the art will understand how to use controls and how to interpret test and control results obtained using methods of the invention.

### Certain Applications and Uses

The teachings of the present disclosure may be used to evaluate, diagnose, or monitor a disease. “Diagnosis” as used herein generally includes a prediction of a subject's susceptibility to a disease or condition (the term “condition” may be used interchangeably herein with the term “disorder”), determination as to whether a subject is presently affected by a disease or condition, prognosis of a subject affected by a disease or condition (e.g., identification of cancerous states, stages of cancer, likelihood that a patient will die from the cancer), prediction of a subject's responsiveness to treatment for a disease or condition (e.g., a positive response, a negative response, no response at all to, e.g., allogeneic hematopoietic stem cell transplantation, chemotherapy, radiation therapy, antibody therapy, small molecule compound therapy) and use of therametrics (e.g., monitoring a subject's condition to provide information as to the effect or efficacy of therapy). For example, a biopsy may be prepared from a cancerous tissue and microscopically analyzed to determine the type of cancer, the extent to which the cancer has developed, whether the cancer will be responsive to therapeutic intervention, etc.

Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above.

As another example, a biopsy may be prepared from a diseased tissue (e.g., kidney, pancreas, stomach, etc.) to determine the condition of the tissue, the extent to which the disease has developed, the likelihood that a treatment will be successful, etc. The terms “treatment,” “treating,” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease. The terms “individual,” “subject,” “host,” and “patient,” are used interchangeably herein and refer to any mammalian subject for whom diagnosis, treatment, or therapy is desired, particularly humans. Examples of diseases that are suitable to evaluation, analysis, diagnosis, prognosis, and/or treatment using the subject methods and systems include, but are not limited to, cancer, immune system disorders, neuropsychiatric disease, endocrine/reproductive disease, cardiovascular/pulmonary disease, musculoskeletal disease, gastrointestinal disease, and the like. Examples of conditions that are suitable to evaluation, analysis, diagnosis, prognosis, and/or treatment using the subject methods and systems include, but are not limited to development, aging, etc.

The teachings of the present disclosure may also be used to evaluate normal tissues, organs, and cells, for example to evaluate the relationships between cells and tissues of a normal tissue sample (e.g., a tissue sample taken from a subject not known to suffer from a specific disease or condition). The subject methods may be used to investigate relationships between cells and tissues during fetal development such as, for example, during development and maturation of the nervous system, as well as to investigate the relationships between cells and tissues after development has been completed, e.g., the relationships between cells and tissues of the nervous systems of a fully developed adult sample.

### EXAMPLES

### Example 1. Experiments for Extracting at Least One Protein from a Biological Sample

An experiment for extracting at least one protein from a biological sample includes obtaining a biological sample including proteins. In some experiments, the biological sample is a tissue sample. In some experiments, the tissue sample is a brain tissue sample.

The experiment includes contacting the biological sample with a photocleavable linker. In some experiments, the photocleavable linker is 1-(1-(4-(acryloyloxy)-5-methoxy-2-nitrophenyl)ethyl) 7-(2,5-dioxopyrrolidin-1-yl) heptanedioate. For example, in some experiments an about 50 μm (mouse coronal) brain slice, is fixed with 4% PFA, and incubated in 0.1 mg/mL of the photocleavable linker. In some experiments, the photocleavable linker is (1-(1-(4-(acryloyloxy)-5-methoxy-2-nitrophenyl)ethyl) 7-(2,5-dioxopyrrolidin-1-yl) heptanedioate)] in PBS. The slice is incubated in 1 mL of the solution for about 6 hours or overnight at room temperature.

Following contacting the biological sample with the photocleavable linker, the experiment includes forming a swellable material around the biological sample. In some experiments, the swellable material is a polyelectrolyte gel. For example, in some experiments the tissue slice is washed with PBS twice and placed in a gelation solution with a paintbrush and incubated for about 30 min at about 4° C. in the dark. The gelation solution is prepared with a ratio of 47:1:1:1 of Stock X, 4HT, TEMED, and APS, respectively. Stock X is a mixture of sodium acrylate, acrylamide, N,N′-methylenebisacrylamide, sodium chloride, 10×PBS, and water. Concentrations and volumes of Stock X, 4HT, TEMED, and APS are as described in Tillberg, P. W., et al., Nat Biotechnol 2016 September; 34 (9): 987-92. Protein-retention expansion microscopy of cells and tissues labeled using is performed standard fluorescent proteins and antibodies. Once prepared, the gelation salutation is placed in ice. A gelation chamber is constructed with No. 1.5 coverslips or Parafilm®, on a slide and topped with a No. 2 coverslip. The brain slice is transferred to the gelation chamber and left to polymerize at about 37° C. for about 2 hours. The tissue is then digested using mild digestion methods, which in some studies is SDS with heat treatment at about 95° C. (as described in Sarkar, D., et al, bioRxiv doi://doi.org/10.1101/2020.08.29.273540; Aug. 30, 2020.), or enzymes such as LysC or trypsin.

After the swellable material is formed, some experiments include expanding the swellable material and separating the proteins contacted with the photocleavable linker. For example, in some experiments, after incubation in the digestion buffer, the gel-tissue is transferred to PBS at about 4° C. The gel-tissue is expanded by adding water and waiting about 20 min three times. To reach higher expansion factors, multiple rounds of expansion are applied as described in Sarkar, D., et al., bioRxiv doi: //doi.org/10.1101/2020.08.29.273540; Aug. 30, 2020. This is achieved by incubating the 1^{st} gels in re-embedding solution and incubating the resulting gel in a 3^{rd} gelation solution

Following expansion of the swellable material, the experiment includes photocleaving the photocleavable linker, of at least one of the proteins, and extracting the at least one protein from the swellable material. For example, in some experiments the gel-tissue is placed under a two-photon microscope, and the laser is directed to a particular region of the slice for photocleavage of the anchored proteins. An excitation wavelength of about 365 nm is used to photocleave when the photocleavable linker includes an o-nitrobenzyl ester moiety at the desired location. To verify whether the photocleavable linker is functional, a large region of the sample is illuminated to cleave proteins out of the gel and exceed the lower detection limit of mass spectrometry (by shining UV light on a large enough area of the sample). Once the laser cleaves these proteins, 50 μL of 1×PBS is added to the gel-tissue mixture to dissolve the proteins and the mixture is run through LC/MS for protein identification. The proteins identified is then mapped back onto the tissue slice to determine the protein content of that particular region.

Once the photocleavable linker is proven to work, a single laser pulse is applied to a single region of the gel-tissue sample. The protein from that single pulse is extracted using gel electrophoresis or stochastic electrotransport.

In some experiments, the extracted protein is identified. Any of the following methods are used in various experiments: nanopores, fluorosequencing, dynamic sequencing, isolating the N-terminal amino acid, or converting the amino acid sequence into nucleic acids.

In some experiments, the spatial relationship of the proteins in the biological sample is determined by iteratively photocleaving and extracting proteins from the expanded swellable material.

### Further Definitions and Construction of Terms

The titles, headings, and subheadings provided herein should not be interpreted as limiting the various aspects of the disclosure. Accordingly, the terms defined herein are more fully defined by reference to the specification in its entirety. All references, patents and patent applications and publications that are cited or referred to in this application are incorporated herein in their entirety herein by reference.

Unless otherwise defined, scientific and technical terms used herein shall have the meanings that are commonly understood by those of ordinary skill in the art. Further, unless otherwise required by context, singular terms shall include pluralities, and plural terms shall include the singular.

In this application, the use of “or” means “and/or” unless stated otherwise. In the context of a multiple dependent claim, the use of “or” refers back to more than one preceding independent or dependent claim in the alternative only.

It is further noted that, as used in this specification and the appended claims, the singular forms “a,” “an,” and “the,” and any singular use of any word, include plural referents unless expressly and unequivocally limited to one referent.

As used herein, the term “about,” means approximately. When the term “about” is used in conjunction with a numerical range, it modifies that range by extending the boundaries above and below the numerical values set forth. Illustratively, the use of the term “about” indicates that values slightly outside the cited values (i.e., plus or minus 0.1% to 10%), which are also effective and safe are included in the value. Numerical ranges recited herein by endpoints include all numbers and fractions subsumed within that range (e.g., 1 to 5 includes 1, 1.5, 2, 2.75, 3, 3.90, 4, and 5).

As used herein, the terms “comprising” (and any form of comprising, such as “comprise,” “comprises,” and “comprised”), “having” (and any form of having, such as “have” and “has”), “including” (and any form of including, such as “includes” and “include”), and “containing” (and any form of containing, such as “contains” and “contain”) are inclusive or open-ended and do not exclude additional, un-recited elements or method steps. Additionally, a term that is used in conjunction with the term “comprising” is also understood to be able to be used in conjunction with the term “consisting of” or “consisting essentially of.”

Method steps described in this disclosure can be performed in any order unless otherwise indicated or otherwise clearly contradicted by context.

For the avoidance of doubt, insofar as is practicable any embodiment of a given aspect of the present disclosure may occur in combination with any other embodiment of the same aspect of the present disclosure. In addition, insofar as is practicable it is to be understood that any preferred or optional embodiment of any aspect of the present disclosure should also be considered as a preferred or optional embodiment of any other aspect of the present disclosure.

What is claimed is:

1. A photocleavable linker capable of embedding proteins of a biological sample, within a swellable material, the photocleavable linker having the formula:
A-B-C
wherein:
A is a protein-binding moiety;
B is a photocleavable moiety; and
C is a swellable material-binding moiety,
wherein the protein-binding moiety comprises 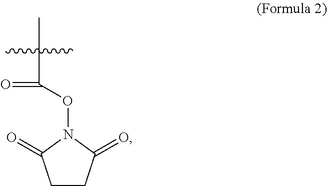 [SMILES: *C(=O)ON1C(=O)CCC1=O]
and the swellable material-binding moiety comprises either: 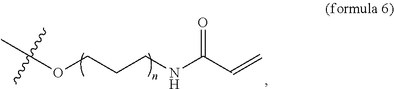
wherein n is any integer capable of being synthesized, or

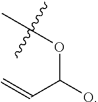

2. The photocleavable linker of claim 1, wherein the protein-binding moiety is capable of binding to an amine group of a protein.

3. The photocleavable linker of claim 1, wherein the protein-binding moiety comprises one or more N-hydroxysuccinimide esters (NHS-esters), or one or more NHS-esters in combination with one or more N,N′-Bis(acryloyl) cystamines, diazirines, and epoxides.

4. (canceled)

5. The photocleavable linker of claim 1, wherein the photocleavable moiety comprises one or more nitrobenzyls, one or more nitrobenzyl derivatives, one or more azobenzenes, one or more ruthenium (II) caged aminosilanes, one or more coumarins, or a combination thereof.

6. The photocleavable linker of claim 5, wherein the one or more nitrobenzyl derivatives comprise one or more ortho-nitrobenzyl esters.

7. The photocleavable linker of claim 5, wherein the one or more ruthenium (II) caged aminosilanes comprise one or more ruthenium-bipyridine-triphenylphosphine caged gamma-aminobutyric acids (RuBi-GABAs).

8. The photocleavable linker of claim 1, wherein the photocleavable moiety is selected from the group consisting of nitrobenzyl, a nitrobenzyl derivative, azobenzene, ruthenium (II) caged aminosilane, and coumarin.

9. The photocleavable linker of claim 1, wherein the swellable material-binding moiety comprises one or more of 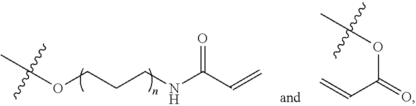
and one or more acryloyls, additional acrylamides, and azides.

10. (canceled)

11. A method for extracting at least one protein from a biological sample, the method comprising:
obtaining a biological sample comprising proteins;
contacting the biological sample with a photocleavable linker, that embeds the proteins of the biological sample, within a swellable material, the photocleavable linker having the formula:
A-B-C
wherein:
A is a protein-binding moiety:
B is a photocleavable moiety; and
C is a swellable material-binding moiety:
wherein the protein-binding moiety comprises 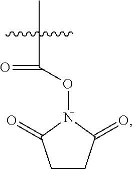 [SMILES: *C(=O)ON1C(=O)CCC1=O]
and the swellable material-binding moiety comprises either: 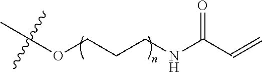
wherein n is any integer capable of being synthesized, or 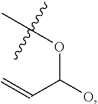
resulting in the biological sample having anchored proteins;
forming a swellable material around the biological sample having the anchored proteins, resulting in the photocleavable linker binding to the swellable material;
expanding the swellable material, thereby separating the anchored proteins;
after expanding the swellable material, photocleaving the photocleavable linker, of at least one of the anchored proteins, resulting in at least one untethered protein; and
extracting the at least one untethered protein.

12. The method of claim 11, wherein the biological sample is a tissue sample, optionally wherein the tissue sample is a brain tissue sample.

13. The method of claim 12, wherein the tissue sample is less than about 200 μm thick or is between about 200 μm and about 500 μm thick.

14-16.

17. The method of claim 11, wherein the swellable material is a swellable polymer, optionally wherein the swellable polymer is a polyelectrolyte gel.

18. (canceled)

19. The method of claim 11, wherein the expanding comprises isotropically expanding the swellable material.

20. The method of claim 11, wherein the photocleaving comprises contacting the photocleavable linker, of the at least one anchored protein, with ultraviolet (UV) light, visible light, or infrared light.

21. The method of claim 20, wherein the UV light has a wavelength of about 300 nm to about 365 nm, optionally wherein the UV light has a wavelength of about 365 nm.

22. (canceled)

23. The method of claim 20, wherein the UV light has a wavelength of about 375 nm to about 450 nm, optionally wherein the UV light has a wavelength of about 427 nm.

24. (canceled)

25. The method of claim 20, wherein the UV light has a wavelength of about 410 nm to about 500 nm, optionally wherein the UV light has a wavelength of about 460 nm.

26-28. (canceled)

29. The method of claim 11, further comprising performing the photocleaving using a two-photon microscope or using a confocal microscope.

30-32. (canceled)

33. The method of claim 11, wherein the extracting is performed using an electric field, optionally wherein the extracting is performed using gel electrophoresis.

34-38. (canceled)

39. A method for protein identification in a biological sample, the method comprising:
the method of claim 11; and
identifying the at least one extracted protein.

40-42. (canceled)
